(19) United States
(12) Patent Application Publication
(10) Pub. No.: US 2011/0077446 A1
(43) Pub. Date: Mar. 31, 2011

(54) Membrane Separation of a Mixture of Close Boiling Hydrocarbon Components

(75) Inventors: Purushottam V. Shanbhag, Claymont, DE (US); Edgar S. Sanders, JR., Newark, DE (US)

(73) Assignee: American Air Liquide, Inc., Fremont, CA (US)

(21) Appl. No.: 12/570,860

(22) Filed: Sep. 30, 2009

(51) Int. Cl. C07C 7/144; B01D 15/08

(52) U.S. Cl. 585/818; 422/261

(57) ABSTRACT

Disclosed are membrane-based systems and methods for the separation of mixtures containing close-boiling hydrocarbon components that overcome certain issues associated with prior art devices.

### BACKGROUND

Olefins, particularly ethylene and propylene, are important chemical feedstocks. Typically they are found in nature or are produced as primary products or byproducts in mixtures that contain saturated hydrocarbons and other components. Before the raw olefins can be used, they usually must be purified from these mixtures. Numerous difficulties have been experienced in this type of separation. Due to their similar relative volatilities, energy-intensive, capital-intensive, multi-trayed distillation columns typically have been used to purify light olefins.

An example of a prior art distillation column for the separation of propylene and propane is illustrated in FIG. 1. In an exemplary method of operation, a raw feedstock of Refinery Grade Propylene (RGP) comprising 70% propylene and 30% propane is introduced to distillation column 105 along feed pipe or pipes 100. Distillation column 105 generally comprises multiple trays, or levels (not shown). In an embodiment, distillation column 105 comprises 135 trays. Operation of a distillation column 105 is primarily determined by a combination of the number of trays and the reflux ratio. In general, the more trays in a distillation column 105, the greater the separation at a constant reflux ratio, but also the greater the capital cost. Conversely, fewer trays can be used if reflux is increased, but operating cost also increases. In the distillation column 105, the lighter components tend to rise and the heavier components tend to sink. The lighter components are extracted along piping 135 and directed to condenser 145 for cooling. Some of the condensed lighter components may be injected back into distillation column 105. Some of the condensed lighter components may be utilized as a propylene product stream 130, such as Commercial Grade Propylene (CGP nominally 93% propylene).

A second stream comprising propane and other heavier components may be extracted from column 105 along piping 115, re-vaporized in vaporizer 125, and injected back into column 105 along piping 110. Some of the second stream may be utilized as a propane product 120 having typically greater than 95% propane.

Column 105 is merely one example of a propylene and propane distillation column. One of ordinary skill in the art would readily understand that many variations are possible.

Typical results from a single distillation column, with a 70% propylene feed, produces a propylene product of 93% purity and a propane product of 95% purity. Accordingly, an improved system would produce results at least as good as a typical prior art distillation column.

The field of art has proposed the use a membrane-based system. U.S. Pat. No. 3,758,603 and U.S. Pat. No. 3,864,418 in the names of Robert D. Hughes and Edward F. Steigelmann describe membranes used in conjunction with metal complexing techniques to facilitate the separation of ethylene from ethane and methane. Similar metal complex and membrane hybrid processes, called facilitated transport membranes, have been described in U.S. Pat. No. 4,060,566 in the name of Robert L. Yahnke and in U.S. Pat. No. 4,614,524 in the name of Menahem A. Kraus. Further membranes have been considered for the separation of olefins from paraffins as an alternative to distillation. However, the separation is difficult largely because of the similar molecular sizes and condensability of the components desired to be separated. The membrane must operate in a hydrocarbon environment under conditions of high pressure and temperature, often resulting in plasticization that may result in a loss of selectivity and/or permeation rate. Such harsh conditions tend to adversely affect the durability and stability of the separation performance of many membrane materials. A membrane system with sufficiently high olefin/paraffin selectivity, high productivity, and sufficient durability in long-term contact with hydrocarbon streams under high pressure and temperature is highly desired.

The art is replete with processes said to fabricate membranes possessing both high selectivity and high fluxes. Without sufficiently high fluxes the required membrane areas required would be so large as to make the technique uneconomical. It is now well known that numerous polymer membranes are much more permeable to polar gases (examples include H₂O, CO₂, H₂S, and SO₂) than to nonpolar gases (N₂, O₂, and CH₄), and that gases of small molecular size (He, H₂) permeate more readily through polymer membranes than large molecules (CH₄, C₂H₆).

However, even considering these difficulties, utilization of membrane separation has taken an important place in chemical technology for use in a broad range of applications. Gas separation has become a major industrial application of membrane technology in the last 15 years. Membrane based technology for the production of nitrogen from air, removal of carbon dioxide from natural gas, and purification of hydrogen now occupy significant shares of the markets for these processes.

Membrane materials and systems for separating olefinic hydrocarbons from a mixture of olefinic and saturated hydrocarbons have been reported, but are not easily or economically fabricated into membranes that offer the unique combination of high selectivity and durability under industrial process conditions to provide economic viability.

For example, several inorganic and polymer/inorganic membrane materials with good propylene/propane selectivity have been studied. However, fabrication of these membranes into practical industrial membranes has proven difficult. Likewise, liquid facilitated-transport membranes have been demonstrated to have attractive separation performance in the lab, but have been difficult to scale up, and have exhibited declining performance in environments typical of an industrial propylene/propane stream.

Solid polymer-electrolyte facilitated-transport membranes have shown to be capable of fabrication into more stable thin film membranes for ethylene/ethane separation. See Ingo Pinnau and L. G. Toy, Solid polymer electrolyte composite membranes for olefin/paraffin separation, J. Membrane Science, 184 (2001) 39-48. However, these membranes are severely limited by their chemical stability in the olefin/paraffin industrial environment.

Carbon hollow-fiber membranes have shown promise in laboratory tests (“Propylene/Propane Separation”, Product Information from Carbon Membranes, Ltd., Israel), but are vulnerable to degradation caused by condensable organics or water present in industrial streams. Moreover, carbon membranes are brittle and difficult to form into membrane modules of commercial relevance.

Membranes based on rubbery polymers typically have olefin/paraffin selectivity too low for an economically useful separation. For example, Tanaka et al. report that the single-gas propylene/propane selectivity is only 1.7 for a polybutadiene membrane at 50° C. (K. Tanaka, A. Taguchi, Jianquiang Hao, H. Kita, K. Okamoto, J. Membrane Science 121 (1996) 197-207) and Ito and Hwang report a propylene/propane selectivity only slightly over 1.0 in silicone rubber at 40° C. (Akira Ito and Sun-Tak Hwang, J. Applied Polymer Science, 38 (1989) 483-490).

Membranes based on glassy polymers have the potential for providing usefully high olefin/paraffin selectivity because of the preferential diffusivity of the olefin, which has a smaller molecular size than the paraffin.

Membrane films of poly(2,6-dimethyl-1,4-phenylene oxide) exhibited pure gas propylene/propane selectivity of 9.1 (Ito and Hwang, Ibid.) Higher selectivity has been reported by Ilinitch et al. (J. Membrane Science 98 (1995) 287-290, J. Membrane Science 82 (1993) 149-155, and J. Membrane Science 66 (1992) 1-8). However, the membrane exhibited plasticization, most likely due to the presence of hydrocarbons.

Polyimide membranes have been studied extensively for the separation of gases. An article by Lee and Hwang discloses a hollow fiber membrane of a polyimide that exhibits a mixed-gas propylene/propane selectivity in the range of 5-8 with low feed pressure (2-4 bar). Kwang-Rae Lee and Sun-Tak Hwang, Separation of propylene and propane by polyimide hollow-fiber membrane module, J. Membrane Science 73 (1992) 37-45.

Krol et al. report a hollow fiber membrane of a polyimide composed of biphenyltetracarboxylic dianhydride and diaminophenylindane which exhibited a pure-gas propylene/propane selectivity of 12. J. J. Krol, M. Boerrigter, G. H. Koops, Polyimide hollow fiber gas separation membranes: preparation and the suppression of plasticization in propane/propylene environments, J. Membrane Science. 184 (2001) 275-286. However, this membrane was plasticized at even low pressures.

Matrimid and P84/Matrimid blend hollow fiber membranes have been shown to plasticize and lose selectivity when exposed to a propane/propylene stream. T. Visser and M. Wessling, Auto and Mutual Plasticization In Single and Mixed Gas C3 Transport Through Matrimid-Based Hollow Fiber Membranes, J. Membrane Science, 312 (2008) pp. 84-96.

Many of these prior art membrane-based techniques of propane/propylene separation utilize a pervaporation mode (liquid feed, gaseous permeate) or gas separation mode (gaseous feed, gaseous permeate). In the pervaporation mode of operation, the liquid feed is “evaporated” to the permeate stream. The latent heat of vaporization is large and a large temperature decrease accompanies the separation. Pervaporation based separation has similarities to propane refrigeration. The high pressure feed permeates the membrane to low pressure. This process is similar to the expansion of propane across an expansion valve in a propylene refrigeration system, resulting in cooling of the permeate stream.

A convenient mathematical method of describing pervaporation is to divide the separation into two steps. The first is evaporation of the feed liquid to form a hypothetical saturated vapor phase on the feed side of the membrane. The second is permeation of this vapor through the membrane to the low pressure permeate side of the membrane. Although no evaporation actually takes place on the feed side of the membrane during pervaporation, this approach is mathematically simple and is thermodynamically equivalent to the physical process.

In pervaporation, transmembrane permeation is typically induced by maintaining the pressure on the permeate side lower than the vapor pressure of the feed liquid. The permeate side pressure can be reduced, for example, by drawing a vacuum on the permeate side of the membrane, by sweeping the permeate side to continuously remove permeating vapor, or by cooling the permeate vapor stream to induce condensation. The feed may also be heated to raise the vapor pressure on the feed side or to at least partially compensate for the temperature drop on permeation.

Certain issues accompany the use of pervaporation, namely, and typically regarded as most important, the temperature decrease causes membrane productivity to decrease and further increase membrane selectivity. This is an inherent problem for pervaporation. It is very difficult to supply sufficient heat at the correct location (face of the membrane) to maintain constant temperature. As a result, productivity decreases exponentially and the number of modules necessary to achieve a desired productivity becomes unacceptably large. Further, the large temperature drop of the feed to the membrane due to the pressure drop often condenses the feed.

At least one prior art patent has identified that temperature has an effect on membrane performance. U.S. Pat. No. 5,679,133 discloses a glassy polymer membrane that is operated at temperatures of less than about 5° C. for separation of gas components. The patent claims a permeation method comprising contacting a first side of a gas separation membrane having a glassy polymer discriminating layer or region with a gas mixture. The polymer cellulose triacetate is expressly excluded. A difference in chemical potential is maintained from the first side of the membrane to a second side of the membrane. At a minimum, one component of the gas mixture relative to a second component selectively permeates from the first side of the membrane through the membrane to the second side of the membrane. The gas mixture contacts the membrane at a temperature of 5° C. or lower. The membrane having a glassy region is selected so that, when using a mixture of 80 mole percent nitrogen and 20 mole percent oxygen as a feed at 30° C. with a pressure of 30 psia on the first side of the membrane and a vacuum of less than 1 mm Hg on the second side of the membrane, the permeability of oxygen in barrers is less than 2000. This patent illustrated that temperatures below ambient could be utilized in permeation procedures.

U.S. Pat. App. Pub. No. 2004/0000513 discloses a plurality of membrane modules disposed in a first product group, a second product group, and optionally one or more intermediate groups used for simultaneous recovery of a very pure permeate product and a desired non-permeate product from a mixture containing organic compounds. Examples of propylene/propane separation are given as simulated by a computer model. The preferred embodiment is a system of three membranes with propylene selectivity over propane. The embodiments disclosed pump feed stock (about 70% propylene/30% propane) in liquid form to a vaporizer and then to a first membrane. The permeate flows through, is compressed, cooled to 200° F. and passed through a second membrane. The permeate is collected as a stream comprising greater than 95% propylene. The non-permeate from the first membrane is passed to a third membrane. Permeate from the third membrane is compressed and passed back through the second membrane. Non-permeate from the third membrane is collected as a propane product.

U.S. Pat. No. 6,986,802 discloses a membrane device comprising multiple perm-selective membranes that are capable of effecting separation of a mixture of two or more compounds in a feed stock which when subjected to appropriately altered conditions of temperature and/or pressure exhibit a bubble point. The enthalpy of the feed stock is adjusted by a heat exchanger. Membrane Efficiency Index of the non-permeate fluid, when withdrawn, is within a range from about 0.5 to about 1.5. The Membrane Efficiency Index is defined as a ratio of the difference between the specific enthalpy of the feed stream entering the membrane device and specific enthalpy of the non-permeate fluid effluent to the difference between the specific enthalpy of the feed stream and the bubble point specific enthalpy of the non-permeate fluid at the non-permeate product pressure and composition. At an MEI of one, the non-permeate is disclosed as being a liquid at its bubblepoint.

U.S. Pat. No. 7,070,694 discloses an apparatus comprising a fractional distillation column and one or more membrane devices utilizing solid perm-selective membranes. The processes are stated as capable of use in simultaneous recovery of a very pure permeate product, a desired non-permeate stream, and one or more distillate products from a fluid mixture containing at least two compounds of different boiling point temperatures. The patent discloses that the cooling effect produced by a membrane when a low pressure permeate is produced from a high pressure feed stock is due to the Joule-Thompson effect. The patent further expresses the need for the incorporation of heat integrated membrane apparatuses with pressure driven membrane separations.

However, these various prior art approaches require excessive capital costs and a multitude of membrane modules.

U.S. Pat. App. Pub. No. 2008/0167512 improves upon the cited prior art. The '512 publication discloses membrane-based systems and methods for separation of propylene and propane that overcome certain issues associated with prior devices and take advantage of a temperature drop across the associated separation membrane. However, the systems and methods still require use of a recycle compressor, which has an associated capital cost as well as operating cost component.

Accordingly, there is a need in the art field for an improved membrane and/or membrane system for the separation of olefins and paraffins that uses fewer membrane modules and requires less capital and operating cost.

### SUMMARY

Disclosed is a process for the membrane-based separation of a mixture of close-boiling hydrocarbon components. A feed stream comprising the mixture of close-boiling hydrocarbon components is fed to a first membrane stage at a temperature and a pressure above a critical point of the mixture. A first permeate stream and a first non-permeate stream are extracted from the first membrane stage. The process may include one or more of the following aspects:
cooling the first permeate stream to produce a liquid product.
the feed stream and the first non-permeate stream each having a viscosity and the ratio of the viscosity of the first non-permeate stream to that of the feed stream being less than 5.
the feed stream and the first non-permeate stream each having a density and the ratio of the density of the first non-permeate stream to that of the feed stream being less than 5.
the feed stream comprising a mixture of propane and propylene.
the first membrane stage having a selectivity for propylene over propane of at least 5.0.
the first membrane stage having a selectivity for propylene over propane of at least 6.5.
the pressure of the feed stream being 700 psi or higher.
the pressure of the feed stream ranging from about 900 psi to about 1100 psi.
the temperature of the feed stream being 96° C. or higher.
feeding the first non-permeate stream to a second membrane stage at a temperature and pressure above the critical point of the first non-permeate stream, extracting a second non-permeate stream, extracting a second permeate stream, and combining the second permeate stream with the feed stream.
the second membrane stage having a selectivity for propylene over propane of at least 3.0.
the second membrane stage having a selectivity for propylene over propane of at least 5.0.
the second non-permeate stream being collected as a liquid or as a two-phase gas/liquid stream having a purity of at least about 95% propane.

Also disclosed is a system for the membrane-based separation of a mixture of close boiling hydrocarbon components. In the system, a feed stream comprising a mixture of close boiling hydrocarbon components is fed to the inlet of a pump. The pump is adapted and configured to pressurize the feed stream to a pressure above a critical pressure of the mixture. The pump's outlet is in fluid communication with the inlet of a first evaporator. The first evaporator is adapted and configured to raise the feed stream's temperature to a temperature above the critical temperature of the mixture. The first evaporator's outlet is in fluid communication with the feed port of the first membrane stage. The first membrane stage further comprises a permeate port and a non-permeate port. The first membrane stage exhibits a selectivity for propylene over propane of at least 6.5. The first membrane stage's non-permeate port is in fluid communication with the feed port of the second membrane stage. The second membrane stage further comprises a permeate port and a non-permeate port. The second membrane stage exhibits a selectivity for propylene over propane of at least 3.0.

The system may further include a second evaporator having an inlet and outlet. The inlet of the second evaporator may be in fluid communication with the non-permeate port of the first membrane stage and the outlet of the second evaporator may be in fluid communication with the feed port of the second membrane stage. The second evaporator is adapted and configured to raise a temperature of a non-permeate stream from the first membrane stage to a temperature above a critical temperature of the non-permeate stream.

### BRIEF DESCRIPTION OF THE FIGURES

For a further understanding of the nature and objects of the disclosed systems and methods, reference should be made to the following detailed description, taken in conjunction with the accompanying drawings, in which like elements are given the same or analogous reference numbers and wherein:

FIG. 1 is a flow diagram showing a prior art distillation column for the separation of propylene and propane.

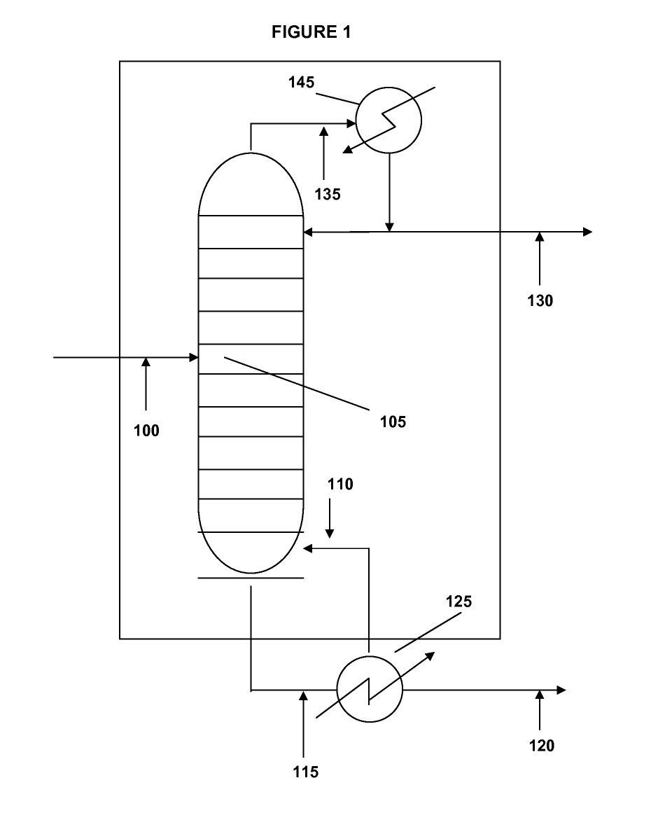

FIG. 2 is a flow diagram showing an embodiment of the current system and method using a membrane separation approach applied to a propylene/propane separation.

FIG. 3 is a flow diagram showing an alternate embodiment of the current system and method applied to a propylene/propane separation.

FIG. 4 is a flow diagram showing a second alternate embodiment of the current system and method applied to a propylene/propane separation.

### DETAILED DESCRIPTION

The following definitions and explanations are meant and intended to be controlling in any future construction unless clearly and unambiguously modified in the following Description or when application of the meaning renders any construction meaningless or essentially meaningless. In cases where the construction of the term would render it meaningless or essentially meaningless, the definition should be taken from Webster's Dictionary, 3rd Edition. Definitions and/or interpretations should not be incorporated from other patent applications, patents, or publications, related or not, unless specifically stated in this specification or if the incorporation is necessary for maintaining validity.

As used herein, a “fluid” is a continuous, amorphous substance whose molecules move freely past one another and that has the tendency to assume the shape of its container, for example, a liquid or a gas.

As used herein, “membrane apparatus” means and refers to flat sheet membranes, spiral wound flat sheet membranes, tubular tube membranes, hollow fiber membranes, and/or other membranes commonly used in industry.

As used herein, “evaporator” means and refers to a heater or an evaporator. In other words, the evaporators utilized herein may be used to raise the temperature and/or change the phase of the stream being processed from liquid to gas or supercritical.

As used herein, “condenser” means and refers to a cooler or a condenser. In other words, the condensers utilized herein may be used to reduce the temperature and/or change the phase of the stream being processed from gas to liquid.

The present disclosure has wide applicability across the art field for systems and methods for the separation of streams containing a mixture of close-boiling hydrocarbon components. The system may operate with either counter current or cross flow membrane bundles. The method relates primarily to a membrane-based system for the separation of streams containing a mixture of close-boiling components that are condensable at ambient conditions by utilizing a feed fluid in its supercritical state.

The details herein were discovered when improving upon the two step process disclosed in U.S. Pat. App. Pub. No. 2008/0167512 by obviating the need for the recycle compressor. Operating the process at a temperature and pressure such that the feed stream fed to the membranes is above the critical point of the feed stream enables the permeate/low pressure side of the membrane to be set at conditions such that the permeate gas phase may be condensed to a liquid by merely lowering the temperature. The resulting liquid permeate may either be available for downstream processing or recycled back to the membrane feed without the need for additional compression. Operation at pressures and temperatures above the critical point also ensures a continuum of densities and viscosities and as a result there are no sharp phase demarcations within the membrane module.

The disclosure therefore provides a method of separating streams containing close-boiling hydrocarbon mixtures, including, but not limited to, mixtures of ethane and ethylene; propane and propene; 1-butene and butane; and 1-butene, butane, and propane. For the purposes of this application, close-boiling hydrocarbon mixtures are defined as mixtures that contain two or more hydrocarbon compounds with at least one of the compounds having a boiling point close to that of at least a second of the compounds at the pressure at which the disclosed system is operated. Close boiling points are usually within about 45° F. (25° C.) or less, preferably within about 27° F. (15° C.) or less, or more preferably within about 9° F. (5° C.) or less.

The disclosure further provides a method of separating nonideal organic streams. For purposes of this application, nonideality is defined by the inability of the ideal gas law to describe the PVT (pressure volume temperature) behavior of the gas. Non ideality for organic gases increases with molecular weight: methane is more ideal than ethane, ethane is more ideal than propane, propane is more ideal than butane, and the like. In most cases, membrane-based separation of nonideal organic gases leads to a temperature decrease on separation. The temperature change increases with increasing non-ideality of the stream. The temperature change arises due to the reduction in pressure experienced by the permeated gas from the initial feed pressure to the final permeate pressure. This temperature change can be approximated by a Joule-Thompson expansion (dT/dP)H.

In general, the disclosed system comprises, at a minimum, a membrane apparatus, a pump, and an evaporator. A supercritical feed containing a mixture of close-boiling compounds is introduced into the disclosed system.

Many of the negative aspects of membrane-based separation of a nonideal fluid stream are minimized in the present disclosure. In the disclosed process, the feed is initially pumped or compressed to a pressure above the critical pressure of the mixture. The pressurized feed stream is then heated in a vaporizer to a temperature higher than the critical temperature of the feed stream. Vaporization of the stream after pressurization reduces the heat required for vaporization relative to that for vaporization prior to pressurization. This is due to the fact that the heat of vaporization decreases with increasing pressure. Pressurization of the feed has additional benefits. Higher pressure reduces total membrane count (i.e., the number of membranes required). Higher pressure increases the pressure-ratio (i.e., feed pressure to the membrane divided by permeate pressure from the membrane). Higher pressure ratio improves separation performance, in various embodiments. Preferably, the pressure difference between the permeate stream and the feed stream is less than about 2,000 psig, more preferably less than about 1,500 psig, and even more preferably less than about 1,300 psig.

The final pressure and temperature of the feed mixture are selected so as to place the feed mixture into its supercritical region, and preferably well into its supercritical region. As a result, the supercritical mixture exhibits properties of both a gas and a liquid. It exhibits the diffusivity of a gas and the solvent properties of a liquid. Initial test results for supercritical mixtures of propane and propylene indicate that the mixture permeates through the membrane like a gas. The supercritical mixture does not exhibit surface tension because the liquid/gas phase boundary no longer exists. Therefore, although the non-permeate or residue stream may exhibit a temperature below its critical temperature, thereby technically forming a liquid, the pressure of the non-permeate remains above its critical pressure, which results in a slight density and viscosity difference between the supercritical feed and the remaining non-permeate stream. In other words, it is believed that utilizing a supercritical feed stream benefits both the membrane and process because the viscosities and densities of the feed and non-permeate streams remain close. To further benefit the membrane and process, the ratio of the viscosity of the non-permeate stream to that of the feed stream should preferably be less than 5, and more preferably less than 2. Additionally, the ratio of the density of the non-permeate stream to that of the feed stream is preferably less than 5, and more preferably less than 3.

Table 1 lists the critical temperature and critical pressure above which the specific exemplary and non-limiting mixtures of close-boiling compounds A, B, and C must be raised to be placed into the supercritical phase, as well as the density and viscosity of the mixture at that temperature and pressure. One of ordinary skill in the art would be capable of calculating the critical temperature and pressure of other mixtures that may also be separated according to the disclosed method.

TABLE 1

| | | | Temp | Press | Density | Viscosity |
|---|---|---|---|---|---|---|
| % (v/v) A | % (v/v) B | % (v/v) C | (° C.) | (psig) | (g/cm³) | (Cp) |
| 50% Ethylene | 50% Ethane | 0% | 21 | 722 | 0.1 | 0.02 |
| 20% Ethylene | 80% Ethane | 0% | 28 | 709 | 0.1 | 0.02 |
| 90% Ethylene | 10% Ethane | 0% | 12 | 719 | 0.1 | 0.02 |
| 81% Propylene | 19% Propane | 0% | 93 | 649 | 0.1 | 0.02 |
| 60% Propylene | 40% Propane | 0% | 93 | 636 | 0.1 | 0.02 |
| 20% Propylene | 80% Propane | 0% | 95 | 613 | 0.1 | 0.02 |
| 5% Propylene | 95% Propane | 0% | 96 | 604 | 0.1 | 0.02 |
| 10% Propylene | 80% 1-Butene | 10% Butane | 142 | 584 | 0.2 | 0.03 |
| 10% Propylene | 80% t2Butane | 10% Butane | 149 | 582 | 0.2 | 0.03 |
| 50% 1-Butene | 50% Butane | 0% | 149 | 571 | 0.2 | 0.03 |
| 10% 1-Butene | 80% Butane | 10% Isobutane | 150 | 546 | 0.1 | 0.02 |
| 10% 1-Butene | 80% C2 Butene | 10% Isobutane | 158 | 596 | 0.2 | 0.03 |
| 30% 1-Butene | 60% Butane | 10% Propane | 146 | 574 | 0.2 | 0.02 |

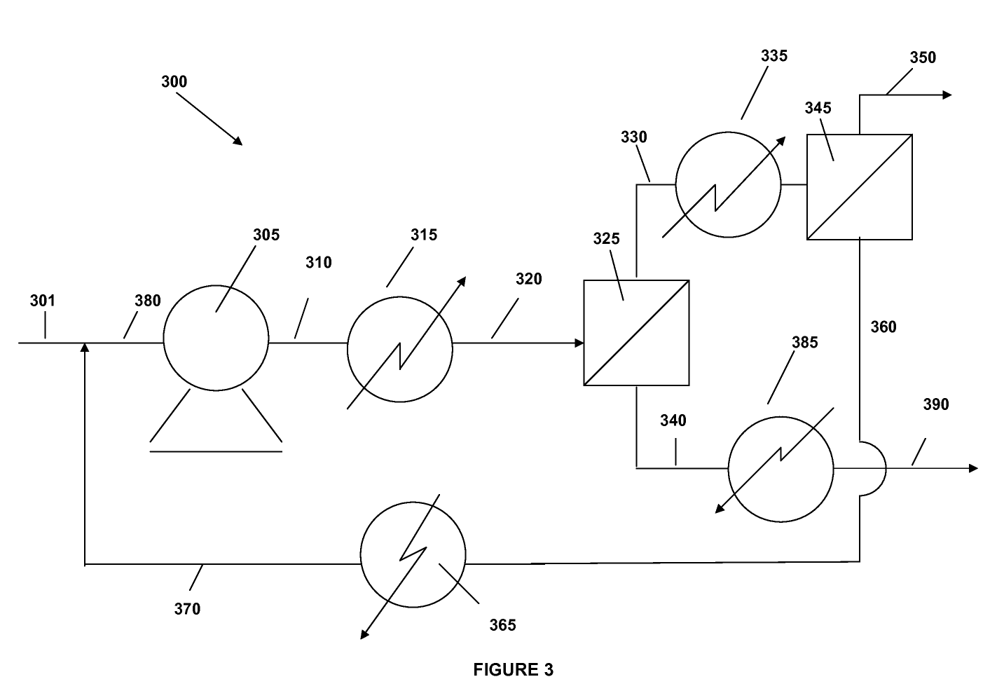

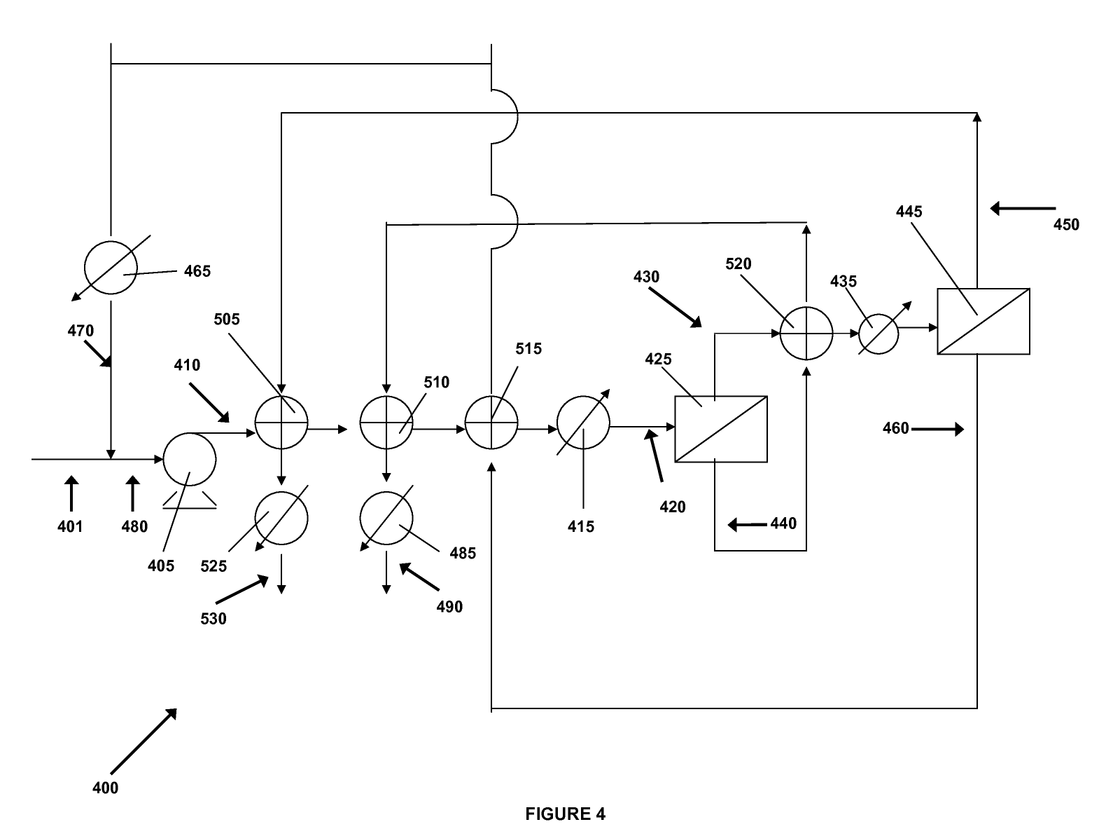

Raising the feed pressure of the mixture above and into the supercritical region significantly mitigates concerns of phase change as the mixture undergoes changes in composition, temperature, and pressure as it is emerges as the non-permeate stream. In addition, raising the permeate pressure provides additional advantages. For example, the permeate stream may undergo a phase change from gas to liquid with only modest cooling. Therefore, the compressors 365 and 395 disclosed in FIG. 3 and 465 in FIG. 4 of U.S. Pat. App. No. 2008/0167512 are not needed. Also the pressure of the permeate stream remains low enough to provide a reasonable pressure ratio across the membrane without effecting the fluid separation. Finally as a liquid pump is used to provide additional compression, the energy requirements may be reduced as much as by 75% when compared to systems that require compressors.

The permeation process causes the temperature of the permeate and non-permeate streams to drop. However, as stated previously, the pressure of the non-permeate stream remains above its critical pressure. Therefore, the density and viscosity of the non-permeate stream remain similar to that of the feed stream. Through optimization of the feed pressure, non-permeate staging, and interstage heating, the temperature difference between the feed stream, the permeate stream, and the non-permeate steam may remain within 30° C., preferably within 20° C., and more preferably within 10° C.

The disclosure contemplates a process for the separation of a mixture containing close-boiling hydrocarbon compounds. The feed stream is at an initial temperature, preferably so that the feed stream is in liquid form. The pressure of the stream is increased to a pressure above the critical pressure of the mixture. The liquid stream is then vaporized and heated to a temperature above the critical temperature of the mixture. The resultant supercritical stream enters the membrane separator. The olefin preferentially permeates through the membrane and the remaining stream (primarily paraffin) is removed as a non-permeate stream. The olefin-enriched permeate may be cooled to form a liquid product. The non-permeate stream may then be reheated to its supercritical phase and passed through another membrane separator wherein the second non-permeate stream is collected as a paraffin product and the second permeate olefin-rich stream is recycled back to the feed stream to increase recovery of the olefin in the olefin product. The second permeate stream requires no further recompression, but simply cooling to be condensed to a liquid and readily mix with the feed stream of the process.

Accordingly, the disclosed process generally comprises the steps of feeding a feed stream comprising a mixture of close-boiling hydrocarbon compounds to a first membrane at a temperature and pressure above the critical point for the mixture, said membrane having a selectivity for olefin as compared to the paraffin of at least 5.0; extracting a permeate olefin enriched stream; cooling the permeate stream; and, recovering the permeate stream as a liquid olefin product stream.

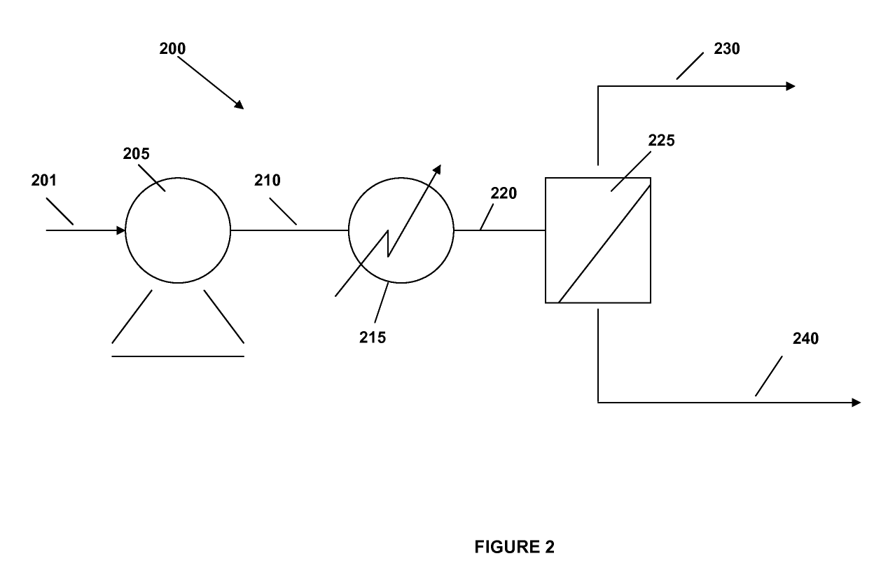

Now referring to FIG. 2, an illustration of a system for the separation of a mixture of close boiling hydrocarbon components is disclosed. System 200 comprises various elements, such as, but not limited to, pump 205, evaporator 215, and membrane stage 225. These elements are interconnected by any means for connection common in the art, such as, but not limited to line(s), piping, valves, and/or the like. For example, in FIG. 2, a line introduces feed fluid 201 to pump 205, a line conveys the pressurized feed fluid 210 to evaporator 215, a line conveys the supercritical feed 220 to membrane stage 225. From membrane stage 225, a line conveys an olefin enriched stream 230 and/or a line conveys a paraffin enriched stream 240.

In a preferred embodiment, during operation of system 200, a feed stock or feed stream 201 comprising at least propylene and propane is introduced or injected into system 200. In this embodiment, the feed stream may be refinery grade propylene (RGP) comprising between about 60% and about 80% propylene, preferably at least about 70% propylene. However, RGP comprising other concentrations of propylene is possible and acceptable. In general, any feed stock comprising any concentration propane and propylene can be utilized in the teachings herein. One of ordinary skill in the art will recognize that this system may also be used to separate different mixtures of close boiling hydrocarbon components.

As disclosed, feed stream 201 is pumped to pressure in pump 205. To separate a mixture comprising at least propylene and propane, the pressure of feed stream 201 is pumped to a pressure of 700 psia or higher prior to introduction to membrane stage 225. Preferably, the pressure of feed stream 201 is pumped to a pressure of about 900 psia to about 1,100 psia prior to introduction to membrane stage 225. In this embodiment, the pressurized feed stream 210 is then vaporized at vaporizer 215 to 96° C. or higher. As a result, the feed stream 220 is fed in its supercritical state to membrane stage 225. One of ordinary skill in the art will recognize that different target temperatures and pressures may be necessary to adapt the system 200 to separate different mixtures of close boiling hydrocarbon components.

Membrane stage 225 may utilize one or more gas separation modules (not shown). In a preferred embodiment, membrane stage 225 is selective for propylene over propane. In this embodiment, any membrane capable of effecting a propylene/propane separation can be used. Membranes capable of operating in a supercritical hydrocarbon environment and effecting a propylene/propane separation are preferred. An example of a membrane capable of operating in a hydrocarbon supercritical environment is a polyimide membrane, and particularly a polyimide membrane made of polymers sold under the tradenames P84 or P84HT from HP Polymers GmbH. Preferred membranes of P84 or P84HT are disclosed in U.S. Pat. No. 7,018,445, titled POLYIMIDE BLENDS FOR GAS SEPARATION MEMBRANES, and U.S. Pat. No. 7,422,623 titled SEPARATION MEMBRANE BY CONTROLLED ANNEALING OF POLYIMIDE POLYMERS. The entire disclosures of these applications are incorporated herein by this reference.

Additional exemplary, non-limiting embodiments and/or disclosures of propane/propylene separation membranes that may be used with the systems and methods disclosed herein are detailed in U.S. Pat. No. 4,374,657; U.S. Pat. No. 4,444,571; U.S. Pat. No. 4,857,078; U.S. Pat. No. 4,952,751; U.S. Pat. Nos. 4,978,430; 5,057,641; U.S. Pat. No. 5,273,572; U.S. Pat. No. 5,326,385; U.S. Pat. No. 5,679,133; U.S. Pat. No. 6,187,196; U.S. Pat. No. 6,187,987; U.S. Pat. No. 6,517,611; U.S. Pat. No. 6,986,802; U.S. Pat. No. 6,693,018; U.S. Pat. No. 7,025,804; and, U.S. Pat. No. 7,070,694, the contents of which are hereby incorporated by reference, as if they were presented herein in their entirety. In general, any membrane apparatus for use in the separation of propylene and propane is capable of use with the present disclosure with varying degrees of performance, as would be readily apparent to one of ordinary skill in the art.

In a preferred embodiment, the selectivity of the propylene/propane membrane for propylene may range from at least about 3.0 to about 20.0. Preferably, the selectivity for propylene ranges from about 5.0 to about 15.0. More preferably, the selectivity for propylene ranges from about 6.5 to about 13.0. Even more preferably, the selectivity for propylene ranges from about 8.0 to about 12.0.

Due to the cooling effect in membrane stage 225, non-permeate stream 230 and permeate stream 240 may be expected to be cooler than feed stream 220. However, it is expected that the temperature and pressure of permeate stream 240 will be such that it remains in its gaseous state. Either or both of stream 230 and/or stream 240 may be further processed as is desired.

The permeate stream 240 may pass from membrane stage 225 and be collected as a gaseous product or cooled to produce a liquid product. In the preferred embodiment, the permeate stream may be used as a chemical-grade propylene product. In this embodiment, the system 200 may be adapted to yield a permeate stream 240 having at least about 93% propylene purity. The percentage recovery of propylene may range from about 50% to about 99%, preferably from about 75% to about 99%, and more preferably from about 85% to about 99%. In the preferred embodiment, the system 200 may be adapted to yield a non-permeate stream 230 having at most about 5% propylene. The percentage recovery of propane from non-permeate stream 230 may range from about 75% to about 99%, preferably from about 80% to about 92%. However, one of ordinary skill in the art will recognize that the percent purity and recovery may vary by design and by composition of the feed stream 201.

Now referring to FIG. 3, an illustration of an alternate embodiment of a system 300 for the separation of a mixture of close boiling hydrocarbon components is disclosed. System 300 comprises various elements, such as, but not limited to, pump 305, first and second evaporators 315, 335, first and optional second condensers 365, 385 (optional), first membrane stage 325, and second membrane stage 345.

These elements are interconnected by any means for connection common in the art, such as, but not limited to line(s), piping, valves, and/or the like. For example, in FIG. 3, a line introduces feed fluid 301 to pump 305, a line conveys the pressurized feed fluid 310 to first evaporator 315, a line conveys the supercritical feed 320 to first membrane stage 325, etc. In a preferred embodiment, during operation of system 300, a feed stock or feed stream 301 comprising at least propylene and propane is introduced or injected into system 300. In this embodiment, the feed stream may be refinery grade propylene (RGP) comprising between about 60% and about 80% propylene, preferably at least about 70% propylene. However, RGP comprising other concentrations of propylene is possible and acceptable. In general, any feed stock comprising any concentration propane and propylene can be utilized in the teachings herein. One of ordinary skill in the art will recognize that this system may also be used to separate different mixtures of close boiling hydrocarbon components.

Feed stream 301 is pumped to pressure in pump 305. The pressure of feed stream 301 is pumped to a pressure above its critical pressure so that, after vaporization, the pressure of the supercritical feed stream 320 remains within its supercritical state. To separate a mixture comprising at least propylene and propane, the feed stream 301 is pumped to a pressure of 700 psia or higher, and more preferably to between about 900 psia and about 1,100 psia. The pressurized feed stream 310 is then vaporized at first evaporator 315 to a temperature of 96° C. or higher, preferably from about 100° C. to about 105° C. As a result, the feed stream 320 is fed in its supercritical state to first membrane stage 325. One of ordinary skill in the art will recognize that different target temperatures and pressures may be necessary to adapt the system 300 to separate different mixtures of close boiling hydrocarbon components.

First membrane stage 325 may utilize one or more gas separation modules (not shown). In a preferred embodiment, the first membrane stage 325 may use membranes similar to those listed above with reference to the membrane stage 225 of FIG. 2 having selectivity for propylene over propane as described above. From first membrane stage 325, a line conveys non-permeate stream 330 and a line conveys permeate stream 340. Permeate stream 340 will exit the first membrane stage 325 at a lower temperature than that at which stream 320 entered first membrane stage 325. However, in a preferred embodiment, it is expected that the temperature and pressure of permeate stream 340 will be such that it remains in its gaseous state. Permeate stream 340 is conveyed to optional second condenser 385 for cooling and conveyed as a hydrocarbon stream 390. In a preferred embodiment, hydrocarbon stream 390 contains substantial quantities of propylene, preferably greater than about 90% propylene, more preferably greater than about 92% propylene, and even more preferably greater than about 93% propylene. Stream 390 may be collected as a product, sent for further processing, used elsewhere in the process, and/or the like.

Non-permeate stream 330 is typically depleted in the olefin component as compared to permeate stream 340 or feed stream 320. However, non-permeate stream 330 is capable of containing some olefin component. Although the percentage of olefin and paraffin has changed in non-permeate stream 330, in a preferred embodiment, the critical temperature and critical pressure for the non-permeate stream 330 remain close to that of feed stream 320. The critical temperature and critical pressure for propylene/propane mixtures only range approximately 4° C. and approximately 60 psig, respectively. As is well known, the pressure of non-permeate stream 330 remains close to that of feed stream 320. Therefore, when the pressure of stream 320 is sufficiently above its critical pressure, additional compression is not required to raise the pressure of non-permeate stream 330 above its supercritical pressure before sending to second membrane 345.

Due to the cooling effect in first membrane stage 325, non-permeate stream 330 may be expected to be cooler than stream 320. In FIG. 3, non-permeate stream 330 is conveyed to a second evaporator 335 to heat stream 330 above its supercritical temperature prior to being fed to second membrane stage 345. In a preferred embodiment, second evaporator 335 heats stream 330 to 96° C. or higher, preferably from about 100° C. to about 105° C. As a result, the non-permeate stream 330 is fed in it supercritical state to second membrane stage 345.

Like the first membrane stage 325, second membrane stage 345 may utilize one or more gas separation modules (not shown). Furthermore, to minimize the temperature change between the non-permeate stream 330, the second non-permeate stream 350, and the second permeate stream 360, one or more combinations of second evaporator 335 and second stage membrane 345 may be utilized in series. For example, a third evaporator (not shown) and a third membrane stage (not shown) may be placed in series with second membrane stage 345 such that they are fed by second non-permeate stream 350 with the resulting permeate stream being mixed with second permeate stream 360 and the resulting non-permeate stream being collected as product. If necessary, to further minimize temperature change, additional evaporator and membrane stage combinations may be utilized in series to process the non-permeate stream.

Second membrane stage 345 is selective for the olefin component over the paraffin component, such that a paraffin-enriched stream 350 is extracted and an olefin-enriched stream 360 is extracted. In a preferred embodiment, second membrane stage 345 may use membranes similar to those listed above for the membrane stage 225 of FIG. 2 having selectivity for propylene over propane as described above. One of ordinary skill in the art will recognize that first and second membrane stages 325 and 345 may utilize the same or different membranes, which may have the same or different selectivities, depending ultimately on the intended purpose of the system 300.

The second permeate stream 360 may pass from second membrane stage 345 and into first condenser 365 for cooling, forming a liquid olefin-enriched stream 370 to be added to stream 301 to form a combined, well-mixed stream 380. One of ordinary skill in the art will recognize how to effectively mix streams 301 and 370. The combination of stream 370 with stream 301 may further improve recovery of olefin in stream 340. As a result, the olefin content of stream 380 may be higher, and the paraffin content lower, than that of stream 301. In a preferred embodiment, the system 300 may be adapted to yield a second permeate stream 360 having less than about 93% propylene purity. The percentage recovery of propylene may range from about 50% to about 99%, preferably from about 75% to about 99%, and more preferably from about 85% to about 99%. However, one of ordinary skill in the art will recognize that the percent purity and recovery may vary by design and by composition of the feed stream 301.

In a preferred embodiment, the system 300 may be adapted to yield a second non-permeate stream 350 having at most about 5% propylene. If the propylene composition of second non-permeate stream 350 is less than 5%, it may be collected as a liquefied petroleum gas (LPG) product. Alternatively, as discussed above, it may be sent to another membrane stage (not shown), set up with components similar to evaporator 335 and second membrane stage 345. The percentage recovery of propane from second non-permeate stream 350 may range from about 50% to about 99%, preferably from about 75% to about 99%, and more preferably from about 80% to about 95%. However, one of ordinary skill in the art will recognize that the percent purity and recovery may vary by design and by composition of the feed stream 301.

Now referring to FIG. 4, an illustration of an alternate embodiment of a system 400 for the separation of a mixture of close boiling hydrocarbon components is disclosed. System 400 discloses a feed stream 401, pump 405, first heat exchanger 505, second heat exchanger 510, third heat exchanger 515, first evaporator 415, first membrane stage 425, fourth heat exchanger 520, second evaporator 435, second membrane stage 445, first condenser 465, second condenser 485, and third condenser 525. In order to maximize heat recovery and minimize operating costs for the system 400, heat exchangers 505, 510, 515, and 520 have been added. One of ordinary skill in the art will recognize that more or fewer heat exchangers may be used without departing from the teachings herein.

These elements are interconnected by any means for connection common in the art, such as, but not limited to line(s), piping, valves, and/or the like. For example, in FIG. 4, a line introduces feed fluid 401 to pump 405; lines convey the pressurized feed fluid 410 through first heat exchanger 505, second heat exchanger 510, third heat exchanger 515, and first evaporator 415; a line conveys the supercritical feed 420 to first membrane stage 425, etc. As in the previous figures, in a preferred embodiment, during operation of system 400, a feed stock or feed stream 401 comprising at least propylene and propane is introduced or injected into system 400. In this embodiment, the feed stream may be refinery grade propylene (RGP) comprising between about 60% and about 80% propylene, preferably at least about 70% propylene. However, RGP comprising other concentrations of propylene is possible and acceptable. In general, any feed stock comprising any concentration propane and propylene can be utilized in the teachings herein. One of ordinary skill in the art will recognize that this system may also be used to separate different mixtures of close boiling hydrocarbon components.

Stream 401 is typically introduced to system 400 as a liquid and pumped to a pressure sufficiently above the critical pressure of stream 401 in pump 405, producing some heat, so that the supercritical feed stream 420 remains within its supercritical range when fed to first membrane stage 425. In a preferred embodiment, the pressure of feed stream 401 is pumped to a pressure so that the pressure of supercritical feed stream 420 is at a pressure of 700 psia or higher prior to introduction to first membrane stage 425. Preferably, the pressure of supercritical feed stream 420 is at a pressure between about 900 psia and about 1,100 psia prior to introduction to first membrane stage 425. One of ordinary skill in the art will recognize that different target pressures may be necessary to adapt the system 400 to separate different mixtures of close boiling hydrocarbon components.

Pressurized feed stream 410 is passed through various heat exchangers for heating by and to provide cooling to other streams. For example, stream 410 may be heated by and provide cooling to streams 450, 440, and/or 460. However, any one or combination of streams 450, 440, and/or 460 may also serve to be cooled by and to heat stream 410.

In a preferred embodiment, first evaporator 415 heats pressurized feed stream 410 to a temperature of 96° C. or higher, preferably from about 100° C. to about 105° C., resulting in supercritical stream 420. One of ordinary skill in the art will recognize that different target temperatures may be necessary to adapt the system 400 to separate different mixtures of close boiling hydrocarbon components. Stream 420 is conveyed to first membrane stage 425. As discussed with respect to FIGS. 2 and 3, first membrane stage 425 may utilize one or more gas separation membrane modules (not shown). In a preferred embodiment, first membrane stage 425 may use membranes similar to those listed above for the membrane stage 225 of FIG. 2 having selectivity for propylene over propane as described above.

Permeate stream 440 will exit the first membrane stage 425 at a lower temperature than that at which stream 420 entered first membrane stage 425. However, it is expected that the temperature and pressure of permeate stream 440 will be such that it remains in its gaseous state. Permeate stream 440 is conveyed to fourth heat exchanger 520, second heat exchanger 510, and second condenser 485 for cooling and conveyed as a liquid hydrocarbon stream 490. In a preferred embodiment, liquid hydrocarbon stream 490 contains substantial quantities of propylene, preferably greater than about 90% propylene, more preferably greater than about 92% propylene, and even more preferably greater than about 93% propylene. Stream 490 may be collected as a product, sent for further processing, used elsewhere in the process, and/or the like.

Non-permeate stream 430 is typically depleted in olefin as compared to permeate stream 440. However, non-permeate stream 430 is capable of containing olefin. As stated with reference to FIG. 3, although the percentage of paraffin and olefin have changed in non-permeate stream 430, in a preferred embodiment, the critical temperature and critical pressure for the non-permeate stream 430 remain close to that of feed stream 420. Therefore, when the pressure of stream 420 is sufficiently above its critical pressure, additional compression is not required to raise the pressure of non-permeate stream 430 above its supercritical pressure before sending to second membrane stage 445.

Due to the cooling effect in first membrane stage 425, however, non-permeate stream 430 may be expected to be cooler than stream 420. The non-permeate stream 430 is passed across fourth heat exchanger 520 with permeate stream 440. Fourth heat exchanger 520 acts to heat stream 430 and cool stream 440. However, little heating of stream 430 may occur due to the volume of the non-permeate stream 430 being greater than that of the permeate stream 440. Alternatively, heating of the non-permeate stream 430 in fourth heat exchanger 520 may range from none at all to more than a minimal amount. If necessary, additional heat may be provided to non-permeate stream 430 by second evaporator 435 to raise the temperature of stream 430 above its supercritical temperature.

Non-permeate stream 430 is conveyed to second membrane stage 445 to produce paraffin-enriched, second non-permeate stream 450 and olefin-enriched, second permeate stream 460. Like first membrane stage 425, second membrane stage 445 may utilize one or more gas separation membrane modules (not shown). Furthermore, to minimize the temperature change between the non-permeate stream 430, the second non-permeate stream 450, and the second permeate stream 460, one or more combinations of second evaporator 435 and second stage membrane 445 may be utilized in series. For example, a third evaporator (not shown) and a third membrane stage (not shown) may be placed in series with second membrane stage 445 such that they are fed by non-permeate stream 450 with the resulting permeate stream being mixed with second permeate stream 460 and the resulting non-permeate stream being collected as product. If necessary, to further minimize temperature change, additional evaporator and membrane stage combinations may be utilized in series to process the non-permeate stream.

Second membrane stage 445 is selective for olefin over paraffin, such that a paraffin-enriched stream 450 is extracted and an olefin-enriched stream 460 is extracted. In a preferred embodiment, second membrane stage 445 may use membranes similar to those listed above for the membrane stage 225 of FIG. 2 having selectivity for propylene over propane as described above. One of ordinary skill in the art will recognize that first and second membrane stages 425 and 445 may utilize the same or different membranes, which may have the same or different selectivities, depending ultimately on the intended purpose of the system 400.

Second non-permeate stream 450 may be at or near supercritical temperature and therefore act to heat stream 410 in first heat exchanger 505. Second non-permeate stream 450 may also be cooled in third condenser 525 and collected as a paraffin-enriched liquid product 530. In a preferred embodiment, the system 400 may be adapted to yield a second non-permeate stream 450 having at most about 5% propylene. If the propylene composition of second non-permeate stream 450 is less than 5%, a LPG product may be collected as stream 530 after cooling in first heat exchanger 505 and third condenser 525. Alternatively, second non-permeate steam 450 may be sent to another membrane stage (not shown), set up in the same manner with components similar to fourth heat exchanger 520, second evaporator 435, and second membrane stage 445. The percentage recovery of propane from second non-permeate stream 450 may range from about 75% to about 99%, preferably from about 80% to about 92%. However, one of ordinary skill in the art will recognize that the percent purity and recovery may vary by design and by composition of the feed stream 401.

Second permeate stream 460 will be pressure depleted and olefin-enriched. At least a portion of stream 460 may act to be cooled by and to provide heat to stream 410 in third heat exchanger 515 and may be cooled in first condenser 465. The resulting liquid olefin-enriched stream 470 may be mixed with feed stream 401 to provide for the additional recovery of olefin. As a result, the olefin content of stream 480 may be higher, and the paraffin content may be lower, than that of stream 401. The combined liquid olefin-enriched stream 470 and feed stream 401 form well-mixed stream 480. One of ordinary skill in the art will recognize how to effectively mix streams 401 and 470. In a preferred embodiment, the system 400 may be adapted to yield a second permeate stream 460 having less than about 93% propylene purity. The percentage recovery of propylene may range from about 50% to about 99%, preferably from about 75% to about 99%, and more preferably from about 85% to about 99%. However, one of ordinary skill in the art will recognize that the percent purity and recovery may vary by design and by composition of the feed stream 401.

Further embodiments may comprise additional membranes as desired. For example, a further membrane may be added to separate olefin from streams 390 and 490, such as to produce, in a preferred embodiment, a Polymer Grade Propylene Product (PGP) product. Alternatively, streams 390 and 490 may be fed to a distillation column for further processing. Further, a different arrangement of heat exchangers, evaporators, compressors, and condensers can be used within the general inventive guidelines of the present invention. The addition of another membrane allows improving olefin purity without the necessity of improving or modifying membrane structure, i.e., membrane selectivity and/or the like. In a preferred embodiment, adding at least one additional membrane increases the purity of propylene to at 99%.

### EXAMPLES

Higher pressure reduces the module count and the higher pressure-ratio (feed pressure to the membrane divided by permeate pressure from the membrane) improves separation performance. Further maintaining the feed in a supercritical state allows operation of the permeate stream at a high enough pressure to achieve its phase change by a simple temperature change.

It has been found that traditional gas separation systems require 12% more overall horsepower and 9.5% more energy for processing RGP than embodiments of the current inventive process. The current inventive process uses ⅓ more pump horsepower than the traditional gas separation approach.

Application of these concepts is illustrated in the following examples. For each of these examples, membrane stages having propylene permeance of 2 and a selectivity of propylene to propane of 8 is assumed.

### Example

Three simulations were performed based upon the system illustrated in FIG. 3, without the optional 385 heat exchanger. In the range of operation, the fugacity coefficient of the feed and non-permeate streams below is estimated to be on the order of approximately 0.5. For an ideal gas, fugacity equals pressure and the fugacity coefficient equals 1. Due to the low fugacity coefficient of the feed stream, the actual driving force across the membrane is roughly half of the pressure-based driving force calculated below. To compensate for this, the bundle counts calculated below should be multiplied by approximately 2 to provide the estimated bundle counts actually needed to practice the disclosed system and method.

The system incorporates membranes 325 and 345 having a propylene permeance of 2 and a propylene to propane selectivity of 8. One of ordinary skill in the art will recognize that membranes 325 and 345 having different selectivities may also be utilized, depending ultimately on the intended purpose of the apparatus. For example, one of ordinary skill in the art will recognize that an increase in the membrane selectivity will result in the need for more membrane modules and that a decrease in the membrane selectivity will result in the need for a larger pump 305.

In each of the three simulations, a feed 301 containing 70.0% (v/v) propylene, 29.8% (v/v) propane, and 0.2% (v/v) iso-butane at a flow of 5326 Nm³/hr, 198.7 psig, and 15.0° C. is fed to pump 305. Pump 305 pressurizes the stream to 1099.0, 899.0, or 699.0 psig respectively. Evaporator 315 vaporizes the pressurized stream 310 to a temperature of 105.0, 100.0, or 100.0° C., respectively. First membrane stage 325 is used to separate the feed into a propylene enriched stream 340 containing approximately 93% propylene and a propane enriched stream 330. The propane enriched stream 330 is reheated by evaporator 335 to 105.0, 100.0, or 100.0° C., respectively, and further processed by second membrane stage 345 into a 95% propane product 350 and a lower pressure propylene-enriched stream 360. Stream 360 is cooled by condenser 365. The resulting propylene-enriched stream 370 is commingled with the feed 301 to produce stream 380. The combined stream 380 is fed to the pump 305. As stated previously, as a result of the addition of liquid propylene-enriched stream 370, the propylene content of the stream 380 fed to the pump 305 may be higher than or equal to, and the propane content lower than or equal to, that of feed stream 301.

Table 2 lists the various properties of the 1099.0 psig pressurization simulation. Table 3 lists the various properties of the 899.0 psig pressurization simulation. Table 4 lists the various properties of the 699.0 psig pressurization simulation.

TABLE 2
Stream Properties for 1099.0 psig pressurization

| | | Feed | Residue | Permeate | Feed | Residue | Permeate |
|---|---|---|---|---|---|---|---|
| Total Bundle Count | | | | | | | 182 |
| Stream # | 301 | 320 | 330 | 340 | 330 | 350 | 360 |
| Flow [Nm3/hr] | 5326 | 14954 | 11051 | 3903 | 11051 | 1391 | 9660 |
| Pressure [psig] | 198.7 | 1099.0 | 1099.0 | 200.0 | 1096 | 1092 | 200 |
| Temp [C.] | 15.0 | 105.0 | 85.8 | 97.9 | 105 | 67 | 82 |
| % Propylene | 70.0 | 72.3 | 65.0 | 93.0 | 65.0 | 4.4 | 73.7 |
| % Propane | 29.8 | 27.5 | 34.8 | 6.9 | 34.8 | 95.2 | 26.1 |
| % iso-Butane | 0.2 | 0.2 | 0.2 | 0.1 | 0.2 | 0.4 | 0.2 |
| Density [g/cm³] | 0.518 | 0.350 | 0.408 | 0.023 | 0.408 | 0.447 | 0.025 |
| Viscosity [cp] | 0.105 | 0.045 | 0.059 | 0.011 | 0.059 | 0.074 | 0.011 |
| Flow [lb/hr] | 22374 | 62754 | 46542 | 16212 | 46542 | 6028 | 40515 |

| | |
|---|---|
| Total Pump + Recycle Energy [KW] | 131 |
| Total Heating Energy [KW] | 3104 |
| Total Cooling Energy [KW] | 3016 |

TABLE 3
Stream Properties for 899.0 psig pressurization

| | | Feed | Residue | Permeate | Feed | Residue | Permeate |
|---|---|---|---|---|---|---|---|
| Total Bundle Count | | | | | | | 291 |
| Stream # | 301 | 320 | 330 | 340 | 330 | 350 | 360 |
| Flow [Nm3/hr] | 5326 | 17926 | 14035 | 3891 | 14035 | 1397 | 12638 |
| Pressure [psig] | 198.7 | 899.0 | 898.5 | 200.0 | 896 | 891 | 200 |
| Temp [C.] | 15.0 | 100.0 | 87.0 | 97.2 | 100 | 68 | 75 |
| % Propylene | 70.0 | 72.9 | 67.3 | 93.0 | 67.3 | 4.6 | 76.3 |
| % Propane | 29.8 | 26.9 | 32.5 | 6.9 | 32.5 | 95.0 | 23.5 |
| % iso-Butane | 0.2 | 0.2 | 0.2 | 0.1 | 0.2 | 0.4 | 0.2 |
| Density [g/cm³] | 0.518 | 0.337 | 0.390 | 0.023 | 0.390 | 0.437 | 0.025 |
| Viscosity [cp] | 0.105 | 0.043 | 0.054 | 0.011 | 0.054 | 0.070 | 0.010 |
| Flow [lb/hr] | 22374 | 75208 | 59045 | 16163 | 59045 | 6053 | 52992 |

| | |
|---|---|
| Total Pump + Recycle Energy [KW] | 123 |
| Total Heating Energy [KW] | 3655 |
| Total Cooling Energy [KW] | 3573 |

TABLE 4
Stream Properties for 699.0 psig pressurization

| | | Feed | Residue | Permeate | Feed | Residue | Permeate |
|---|---|---|---|---|---|---|---|
| Total Bundle Count | | | | | | | 657 |
| Stream # | 301 | 320 | 330 | 340 | 330 | 350 | 360 |
| Flow [Nm3/hr] | 5326 | 27393 | 23510 | 3884 | 23510 | 1413 | 22097 |
| Pressure [psig] | 198.7 | 699.0 | 697.9 | 200.0 | 695 | 691 | 200 |
| Temp [C.] | 15.0 | 100.0 | 98.4 | 99.9 | 100 | 52 | 95 |
| % Propylene | 70.0 | 74.1 | 70.9 | 93.1 | 70.9 | 4.5 | 80.5 |
| % Propane | 29.8 | 25.7 | 28.9 | 6.8 | 28.9 | 95.1 | 19.3 |
| % iso-Butane | 0.2 | 0.2 | 0.2 | 0.1 | 0.2 | 0.4 | 0.2 |
| Density [g/cm³] | 0.518 | 0.167 | 0.199 | 0.023 | 0.199 | 0.461 | 0.023 |
| Viscosity [cp] | 0.105 | 0.020 | 0.023 | 0.011 | 0.023 | 0.080 | 0.011 |
| Flow [lb/hr] | 22374 | 114860 | 98729 | 16131 | 98729 | 6123 | 92606 |

| | |
|---|---|
| Total Pump + Recycle Energy [KW] | 135 |
| Total Heating Energy [KW] | 6084 |
| Total Cooling Energy [KW] | 6029 |

As discussed previously, although the non-permeate stream exhibits a temperature below its critical temperature, thereby forming a liquid, the density and viscosity of the non-permeate stream remain close to that of the supercritical feed stream, providing less concern about phase change. The system and method disclosed operate most efficiently when the ratios of the viscosity and the density of the non-permeate stream to that of the feed stream approach 1.

At the first membrane stage of the 1099.0 psig pressurization system, the viscosity ratio between the residue and feed stream is 1.3 and the density ratio between the same is 1.16. The viscosity ratio between the residue and feed stream at the first membrane stage of the 899.0 psig pressurization system is 1.2 and its density ratio for the same is 1.16. Similarly, the viscosity ratio between the residue and feed stream at the first membrane stage of the 699.0 psig pressurization system is 1.2 and its density ratio for the same is 1.19.

At the second membrane stage of the 1099.0 psig pressurization system, the viscosity ratio between the residue and feed stream is 1.2 and the density ratio between the same is 1.10. The viscosity ratio between the residue and feed stream at the first membrane stage of the 899.0 psig pressurization system is 1.3 and its density ratio for the same is 1.12. In contrast, the viscosity ratio between the residue and feed stream at the first membrane stage of the 699.0 psig pressurization system is 3.5 and its density ratio for the same is 2.32.

As can be seen, the 1099.0 psig pressurization provides the best results. The 1099.0 psig pressurization apparatus utilizes less membrane modules than the 899.0 and 699.0 examples (see total bundle count). It also utilizes less heating and cooling energy. Finally, the 1099.0 psig pressurization exhibits a smaller viscosity and density ratio between the residue and feed streams of the second membrane stage, which is beneficial to operation of the disclosed membranes and process.

It will be understood that many additional changes in the details, materials, steps, and arrangement of parts, which have been herein described and illustrated in order to explain the nature of the invention, may be made by those skilled in the art within the principle and scope of the invention as expressed in the appended claims. Thus, the present invention is not intended to be limited to the specific embodiments in the examples given above and/or the attached drawings.

What is claimed is:

1. A process for the membrane-based separation of a mixture of close-boiling hydrocarbon components, said process comprising the steps of:
feeding a feed stream comprising the mixture of close-boiling hydrocarbon components to a first membrane stage at a temperature and a pressure above a critical point of the mixture;
extracting a first permeate stream from the first membrane stage; and
extracting a first non-permeate stream from the first membrane stage.

2. The process of claim 1, further comprising the step of cooling the first permeate stream to produce a liquid product.

3. The process of claim 1, wherein the feed stream and the first non-permeate stream each has a viscosity and the ratio of the viscosity of the first non-permeate stream to that of the feed stream is less than 5.

4. The process of claim 1, wherein the feed stream and the first non-permeate stream each has a density and the ratio of the density of the first non-permeate stream to that of the feed stream is less than 5.

5. The process of claim 1, wherein the feed stream comprises a mixture of propane and propylene.

6. The process of claim 5, wherein the first membrane stage has a selectivity for propylene over propane of at least 5.0.

7. The process of claim 6, wherein the first membrane stage has a selectivity for propylene over propane of at least 6.5.

8. The process of claim 5, wherein the pressure of the feed stream is 700 psi or higher.

9. The process of claim 8, wherein the pressure of the feed stream ranges from about 900 psi to about 1100 psi.

10. The process of claim 5, wherein the temperature of the feed stream is 96° C. or higher.

11. The process of claim 1, further comprising the steps of:
feeding the first non-permeate stream to a second membrane stage at a temperature and pressure above the critical point of the first non-permeate stream;
extracting a second non-permeate stream;
extracting a second permeate stream; and
combining the second permeate stream with the feed stream.

12. The process of claim 11, wherein the feed stream comprises a mixture of propane and propylene.

13. The process of claim 12, wherein the second membrane stage has a selectivity for propylene over propane of at least 3.0.

14. The process of claim 13, wherein the second membrane stage has a selectivity for propylene over propane of at least 5.0.

15. The process of claim 14, wherein the second non-permeate stream is collected as a liquid or as a two-phase gas/liquid stream having a purity of at least about 95% propane.

16. A system for the membrane-based separation of a mixture of close boiling hydrocarbon components comprising, in combination:
a feed stream comprising a mixture of close boiling hydrocarbon components;
a pump having an inlet and outlet, the inlet being adapted to receive the feed stream, the pump being adapted and configured to pressurize the feed stream to a pressure above a critical pressure of the mixture;
a first evaporator having an inlet and outlet, the inlet of the first evaporator being in fluid communication with the outlet of the pump, the first evaporator being adapted and configured to raise the feed stream's temperature to a temperature above a critical temperature of the mixture;
a first membrane stage, the first membrane stage having a selectivity for propylene over propane of at least 6.5, a feed port, a non-permeate port, and a permeate port, the first membrane stage feed port being in fluid communication with the outlet of the first evaporator; and,
a second membrane stage, the second membrane stage having a selectivity for propylene over propane of at least 3.0, a feed port, a non-permeate port, and a permeate port, the second membrane stage feed port being in communication with the first membrane stage non-permeate port.

17. The system of claim 16, further comprising a second evaporator having an inlet and outlet, the inlet of the second evaporator being in fluid communication with the non-permeate port of the first membrane stage, the outlet of the second evaporator being in fluid communication with the feed port of the second membrane stage, the second evaporator being adapted and configured to raise a temperature of a non-permeate stream from the first membrane stage to a temperature above a critical temperature of the non-permeate stream.
